(19) United States
(12) Patent Application Publication
(10) Pub. No.: US 2024/0199557 A1
(43) Pub. Date: Jun. 20, 2024

(54) METHODS OF MAKING 1-BENZYL-4-PHENYL-1H-1,2,3-TRIAZOLE (BPT) AND BPT COATED STRUCTURES

(71) Applicant: BOARD OF REGENTS, THE UNIVERSITY OF TEXAS SYSTEM, Austin, TX (US)

(72) Inventors: Brendy Carolina Rincon Troconis, San Antonio, TX (US); Oladis M. Troconis de Rincon, San Antonio, TX (US); Leonardo X. Alvarez, San Pedro (CR); Loreto Dacio, San Antonio, TX (US); Jorge Esribano, San Antonio, TX (US)

(21) Appl. No.: 18/537,337

(22) Filed: Dec. 12, 2023

Related U.S. Application Data
(60) Provisional application No. 63/431,888, filed on Dec. 12, 2022.

(51) Int. Cl. C07D 249/04; C09D 5/08

(52) U.S. Cl. CPC C07D 249/04; C09D 5/08

(57) ABSTRACT

The present disclosure provides for methods of making 1-Benzyl-4-Phenyl-1H-1,2,3-triazole (BPT) and structures having a coating of BPT.

### CROSS REFERENCE TO RELATED APPLICATIONS

This application claims the benefit of U.S. Provisional Application No. 63/431,888 entitled “METHODS OF MAKING 1-BENZYL-4-PHENYL-1H-1,2,3-TRIAZOLE (BPT) AND BPT COATED STRUCTURES” and filed on Dec. 12, 2022, which is incorporated herein by reference in its entirety.

### BACKGROUND

Concrete has a compressive strength that is extremely useful in the design of structures but lacks the tensile strength needed for construction applications. This limitation is predominantly circumvented by the addition of embedded steel reinforcement bars in the concrete. Reinforced concrete is very durable, and it is commonly used in much of the infrastructure in the U.S. and all over the world. Its inclusion in critical infrastructure should highlight the importance of keeping reinforced concrete components from failure, and one such failure is the corrosion of the embedded steel. The cement in the concrete produces a very alkaline environment (pH>12.5), allowing the embedded steel to passivate. As the concrete cures, pores are developed and allow penetration of air and water, allowing the transport of CO₂, O₂, and chloride ions inside the concrete. The most common mechanism for corrosion of the reinforcement is caused by the presence of chloride ions, which can, in sufficient amounts, cause localized breakdown of the passive oxide layer.

One method of preventing or reducing the effects of chlorides and slow down corrosion is using chemical admixtures that inhibit corrosion. These inhibitors can be organic or inorganic and can be introduced as an admixture to the cement, applied to the surface post curing, or come from a microcapsule triggered by a pH change. To date, the longest used inorganic inhibitor that has been evaluated enough to justify its continuing commercial use to control the corrosion of reinforced concrete is Calcium Nitrite (Ca(NO₂)₂). Calcium nitrite has produced the best results in alkaline environment, even in cracked concrete, but when used in insufficient quantities, it can aggravate the corrosion performance by pitting. Therefore, there is a need for a corrosion inhibitor (CI) that, in addition to protecting steel in chloride containing environments, is environmentally friendly and does not detrimentally affect the concrete mechanical properties.

In recent years, organic corrosion inhibitors (OCIs) have been gaining widespread use in reinforced concrete. OCIs inhibit corrosion by either chemisorption, physisorption mechanism, or both.

Despite the commercial use, studies evaluating OCIs, such as amines and alkanolamines, found mixed results concerning the effectiveness of these compounds. Furthermore, despite the classification as organic, OCIs may be synthetic and toxic, causing harm to the environment, similar to the inorganic CI Ca(NO₂)₂. Therefore, there is a need for the development of environmentally friendly inhibitors, commonly known as green CIs (GCIs). It can be beneficial if the GCIs, in addition to inhibiting corrosion, are low-cost, eco-friendly during synthesis and use, and harmless once disposed.

### SUMMARY

The present disclosure provides for methods of making 1-Benzyl-4-Phenyl-1H-1,2,3-triazole (BPT), structures having a coating of BPT, and the like.

The present disclosure provides for a method of making 1-Benzyl-4-Phenyl-1H-1,2,3-triazole (BPT), comprising: mixing t-BuOH, water, benzyl bromide and sodium azide to form a first mixture; mixing phenylacetylene, ascorbic acid, sodium hydroxide, and pentahydrated copper sulfate with the first mixture, wherein a precipitate is formed; separating the precipitate, wherein the precipitate is BPT. In an aspect, the phenylacetylene, ascorbic acid, sodium hydroxide, and pentahydrated copper sulfate are mixed to form a second mixture prior to mixing with the first mixture.

The present disclosure provides for a method of making 1-Benzyl-4-Phenyl-1H-1,2,3-triazole (BPT) comprising the following reaction mechanism:

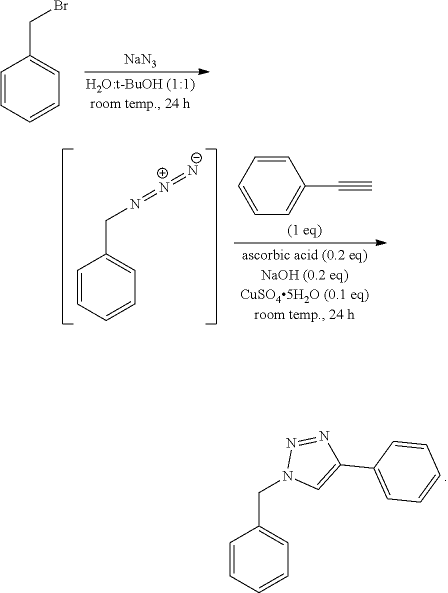

The present disclosure provides for a structure comprising: a steel structure having a coating of 1-Benzyl-4-Phenyl-1H-1,2,3-triazole (BPT) disposed on the surface, wherein the BPT was made using the method described above and herein.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an improved synthesis of 1-Benzyl-4-Phenyl-1H-1,2,3-triazole (BPT) involving two sequential chemical reactions in the same reaction flask.

FIG. 2 illustrates an average open circuit potential (OCP) and solution pH (measured after tests) measurements for different conditions, with and without 2 M NaCl.

FIG. 3 illustrates the corrosion rate (CR) of steel rebar calculated for different conditions, with and without 2 M NaCl.

FIG. 4 illustrates the correlation of CR and corrosion inhibitor (CI) efficiency for different BPT concentrations in synthetic pore solution (SPS) solutions with 2 M NaCl.

FIG. 5 illustrates Anodic Cyclic Polarization (CYP) tests for carbon steel rebar in different SPS solutions with BPT concentrations after exposure in solutions from Table 2 without NaCl.

FIG. 6 illustrates CYP tests for carbon steel rebar in different SPS solutions with BPT concentrations after exposure in solutions from Table 2 in presence of 2 M NaCl.

FIG. 7 illustrates Rp and OCP values for steel rebar immersed in solutions with lower (3 mM BPT+NMP), and same ((Adj.) 3 mM BPT+NMP) initial hydroxide concentration with respect to SPS for 3 mM BPT, and control (SPS) in presence of Cl⁻.

FIG. 8 illustrates a comparison of CYP measurements of SPS, aggressive environment with optimum BPT (3 mM BPT+NMP), and environment with adjusted hydroxide concentration with optimum BPT concentration ((Adj.) 3 mM BPT+NMP) in presence of Cl⁻.

FIG. 9 illustrates X-Ray powder diffraction (XRD) patterns of polished carbon steel discs before and after a 4-week immersion in SPS and in the presence of different additives.

FIGS. 10A-10B illustrate XRD patterns of polished carbon steel discs after 4, 8, and 12-week immersion in SPS (10A) and after a 12-week immersion in SPS (10B) and 2 weeks after the addition of solid NaCl (10B).

FIGS. 11A-11D illustrate corrosion morphology of carbon steel showing: freshly polished surface (11A), after 1-week immersion in SPS (11B), two weeks after the addition of NaCl (11C), and disc 4 (11D) after a month immersion in SPS containing NaCl, NMP, and BPT.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, tribology, and the like, which are within the skill of the art.

The following examples are put forth so as to provide those of ordinary skill in the art with a complete disclosure and description of how to perform the methods and use the compositions, methods, and materials disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DISCUSSION

Embodiments of the present disclosure provide for methods of making 1-Benzyl-4-Phenyl-1H-1,2,3-triazole (BPT), structures having a coating of BPT, and the like. The present disclosure provides the following advantages: a “green” method for producing BPT, which avoids the use of certain solvents and reactants that can be problematic and an effective coating for structure such as steel to reduce the deleterious effects of corrosion.

In an aspect, the present disclosure provides for an effective method of making 1-benzyl-4-phenyl-1H-1,2,3-triazole (BPT), where BPT was evaluated as a potential green organic corrosion inhibitor to reduce the effects of corrosion on the reinforcing steel in concrete. The BPT inhibitor was assessed in synthetic pore solution (e.g., SPS, 8.33 g/L of NaOH+23.3 g/L of KOH+2.0 g/L of Ca(OH)₂, pH=13.6) in presence of 2 M NaCl and following a carbonation free and rebar passivation stabilization protocol prior Cl⁻ addition, similar to ASTM G180. A base solution (2.35 mg/mL) of BPT in N-methyl-2-pyrrolidone (NMP) was prepared due to the limited solubility of BPT in water.

Electrochemical techniques including open circuit potential, linear polarization resistance and cyclic polarization curves were conducted to assess the performance of the inhibitor. The results showed that the BPT inhibitor effectively reduces the corrosion rate of the steel rebar. In a particular aspect, the BPT concentration in SPS with 2M NaCl was 3 mM BPT had an efficiency of 85.2%. Furthermore, XRD showed evidence of an adsorption mechanism by which BPT controls the corrosion rate of steel in 2 M NaCl. Additional details are provided in the Example.

In general, the method of making 1-benzyl-4-phenyl-1H-1,2,3-triazole (BPT) can include mixing t-BuOH, water, benzyl bromide and sodium azide to form a first mixture. Then phenylacetylene, ascorbic acid, sodium hydroxide, and pentahydrated copper sulfate are mixed with the first mixture to form a precipitate. Subsequently, the precipitate is separated. The precipitate is then rinsed and dried. The precipitate is BPT. The yield for producing BPT is greater than 90%, is about 95% or more, or is about 97% or more, or about 90% to 100%, about 95% to 100%, or about 97% to 100%.

More particularly, the method of making BPT can include mixing t-BuOH, water, benzyl bromide and sodium azide to form a first mixture. The first mixture is mixed for at least 1 hour, about 1 to 48 hours, or about 12 to 24 hours. A second mixture is formed by mixing phenylacetylene, ascorbic acid, sodium hydroxide, and pentahydrated copper sulfate. The second mixture is mixed for at least 1 hour, about 1 to 48 hours, about 12 to 24 hours. The first mixture and the second mixture are mixed to form a third mixture, where the third mixture includes a solution and precipitate. The third mixture is mixed for at least 1 hour, about 1 to 48 hours, about 12 to 24 hours. The precipitate is separated from the solution. The precipitate is BPT and the yield for producing BPT is greater than 90%, is about 95% or more, or is about 97% or more, or about 90% to 100%, about 95% to 100%, or about 97% to 100%.

In a particular aspect, the method can be described by the following scheme:

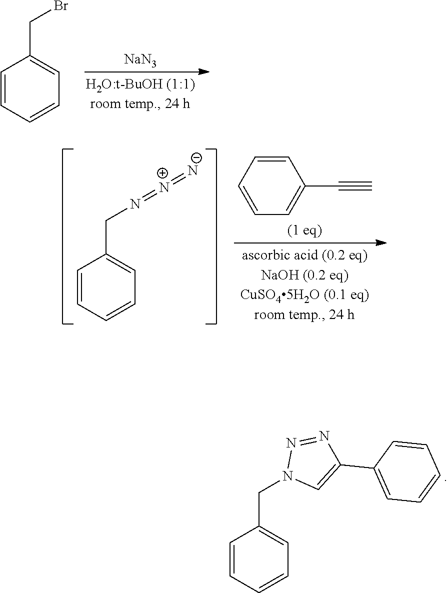

The present disclosure also provides for structures coated with BPT. In particular, the BPT is coated on the surface of the structure. The structure can be steel or other material that may be subject to corrosion. The BPT of the BPT coating is made using the methods described above and herein.

In an aspect, the steel structure having the BPT coating is disposed within a concrete material, such as reinforced concrete. The steel structure can be rebar. The BPT coating can have a thickness of on the nanometer scale to millimeter scale and to the centimeter scale if desired. In particular, the thickness can be about 10 nm to 10 cm, about 100 nm to 1 cm, about 500 nm to 500 mm, about 1 mm to about 10 mm, and the like. The BPT coating thickness may not be identical in all regions of the structure. In an aspect, the coating of BPT has the characteristic of inhibiting the corrosion of the steel structure in SPS with 2 M NaCl at concentrations higher than 2 mM. But, the optimal concentration may be about 3 mM BPT with an efficiency of about 85.2%. Additional details are provided in Example 1.

### EXAMPLE

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

### 1. Introduction

Concrete has a compressive strength that is extremely useful in the design of structures but lacks the tensile strength needed for construction applications [1]. This limitation is predominantly circumvented by the addition of embedded steel reinforcement bars in the concrete. Reinforced concrete is very durable, and it is commonly used in much of the infrastructure in the U.S. and all over the world [2]. It is utilized in highway bridges, commercial and residential buildings, and critical structures such as dams and nuclear facility safety structures [3]. Its inclusion in critical infrastructure should highlight the importance of keeping reinforced concrete components from failure, and one such failure is the corrosion of the embedded steel.

The cement in the concrete produces a very alkaline environment (pH>12.5), allowing the embedded steel to passivate [1]. As the concrete cures, pores are developed and allow penetration of air and water, allowing the transport of CO₂, O₂, and chloride ions inside the concrete. The CO₂ could reduce the pH through carbonation, leading to corrosion initiation, but the most common mechanism for corrosion of the reinforcement is caused by the presence of chloride ions, which can, in sufficient amounts, cause localized breakdown of the passive oxide layer [4-8].

One method of preventing or reducing the effects of chlorides and slow down corrosion is using chemical admixtures that inhibit corrosion. These inhibitors can be organic or inorganic, and can be introduced as an admixture to the cement, applied to the surface post curing, or more recently from a microcapsule triggered by a pH change [9]. This can be accomplished through different methods such as, slowing the ingress of chlorides, or chemically binding with the chloride ions, or increasing the chloride threshold required for corrosion initiation, or by reducing the corrosion rate (CR) of the embedded metal once corrosion initiates [4, 8, 10].

To date, the longest used inorganic inhibitor that has been evaluated enough to justify its continuing commercial use to control the corrosion of reinforced concrete is Calcium Nitrite (Ca(NO₂)₂) [11-17]. However, its effectiveness is achieved if the Cl⁻/NO₂⁻ ratio is lower than 1 [15]. Calcium nitrite has produced the best results in alkaline environment, even in cracked concrete [11, 18]. When used in insufficient quantities, it can aggravate the corrosion performance by pitting [12]. A study [12] of the effect of mixing this inhibitor with ZnO in equal proportions (2% on cement base, each one), lead to a better performance. However, there are no long-term results in real structures, which is the same case for calcium nitrite. Therefore, there is a strong need for corrosion inhibitor (CI) that, in addition to protecting steel in chloride containing environments, is environmentally friendly and does not detrimentally affect the concrete mechanical properties.

In recent years, organic corrosion inhibitors (OCIs) have been gaining widespread use in reinforced concrete [8, 19, 20]. OCIs inhibit corrosion by either chemisorption, physisorption mechanism or both. They attach to the surface through a functional group that may have atoms with non-bonding electrons (N, O, S or P), multiple bonds (pi electrons) or both. Once on the metal surface, they promote the growth and maintenance of intrinsic passive films [21-23]. Both the anodic and cathodic processes have been shown to be suppressed, categorizing most OCIs as mixed inhibitors [22]. Amines and alkanolamines are increasingly becoming a commercially available OCI due to high water solubility and low influence on the physical properties of concrete [8, 22]. Despite the commercial use, studies evaluating amines and alkanolamines found mixed results concerning the effectiveness of these compounds [22-24]. Notable compounds studied were triethanolamine (TEA), monoethanolamine (MEA), dimethylethanolamine (DMEA) alkanolamines, and triethylenetetramine (TETA) amines. DMEA was found to negatively affect the workability of concrete mixes at the concentrations needed to effectively inhibit corrosion, while MEA was considered to have high volatility, thus causing unreproducible results. Of the tested amines and alkanolamines, TETA and TEA showed the best results.

Despite the classification as organic, the CIs may be synthetic and toxic causing harm to the environment, similar to the inorganic CI Ca(NO₂)₂[25]. For instance, the amine TEA has been noted as an occupational health hazard causing visual disruption [26] and the production of other inhibitors can produce harmful biproducts [27, 28]. Therefore, there is a need for the development of environmentally friendly inhibitors, commonly known as green CIs (GCIs) [8]. These GCIs, in addition to inhibiting corrosion, must be low-cost, eco-friendly during synthesis and use, and be harmless once disposed. Examples of GCIs include red mud, rare earths, organic polymers, plant extracts from natural stems, leaves, and seeds. Among these, plant extracts are of great interest. For example, Bambusa arundinacea extracts have been seen to enhance the passivation layer and outperformed Ca(NO₂)₂ in inhibiting reinforced concrete exposed to seawater, while also being biodegradable, affordable and of low environmental impact [27, 28]. Similarly, other materials from organic origin but synthetic, can be used when considering environmental impact in the mitigation of corrosion in reinforced concrete. 1-Benzyl-4-Phenyl-1H-1,2,3-triazole (BPT) is one such compound that has been synthesized with green principles in mind [29]. This synthetic organic molecule, has already been explored as a CI on A36 mild steel in acidic environments (1 M HCl), resulting in reduction of the corrosion kinetics from 2.311 mm/year to 0.429 mm/year in 1M HCl at 298 K.

BPT is a 1,2,3-triazole derivative that has been utilized as an OCI on copper and on A36 mild steel [29, 30]. It is a synthetic organic compound that contains the necessary active centers (N-atom and multiple bonds) needed for the adsorption of the chemical onto the metallic surface and the passivation of the substrate [8]. Previous iterations of the synthesis of BPT required several synthetic steps, utilized hard-to-get starting materials or used toxic additives. The use of these procedures disqualified the classification of BPT as a GCI using the criterion mentioned earlier (low-cost, eco-friendly during synthesis and use, and be harmless once disposed, in addition to inhibiting corrosion [8, 27]).

However, the method used in this example for the preparation of BPT follows a protocol that aims to reduce the use of toxic additives, resulting in a product that reduces environmental harm throughout its life cycle. As BPT has proven to be effective as a CI for Cu and mild steel in acidic media [27, 28] and there is some evidence of the application of triazoles as CIs for the protection of Al in chloride-containing alkaline solutions [31], the BPT as a GCI on carbon steel rebars was evaluated in synthetic pore solution (SPS) in the presence of chlorides. This SPS mimics the capillary pore chemical conditions found in reinforced concrete components by containing alkalis commonly found under this condition and has already been proven as a suitable testing solution [7, 32-35]. Several conditions were tested, each containing various concentrations of the BPT inhibitor.

This Example presents the synthesis and evaluation of the effectiveness of BPT as a possible GCI for carbon steel rebars in SPS in presence of 2 M NaCl. For this purpose, different electrochemical techniques, including open circuit potential, linear polarization resistance and cyclic polarization were used to assess the GCI. This information was complemented with film characterization using XRD. This work will further aid in the development and study of GCI methods for use in reinforced concrete, in place of other traditional corrosion inhibitors, which are not environmentally friendly.

### 2. Experimental Procedure

### 2.1 Materials

### Substrate

Experiments were carried out on the cross section of steel cylinders machined from #3 carbon steel reinforcing bars with chemical composition shown in Table 1.

TABLE 1
Chemical composition (wt. %) of rebar material

| C | Mn | P | S | Si | Cu | Cr | Ni | Mo | V | Cb | Sn | Al | Fe |
|---|---|---|---|---|---|---|---|---|---|---|---|---|---|
| 0.41 | 0.57 | 0.013 | 0.051 | 0.16 | 0.35 | 0.19 | 0.22 | 0.092 | 0.003 | — | 0.012 | — | Bal. |

### BPT Synthesis

This synthesis is based on a previous work [29], but it has been improved from the point of view of green chemistry [36]. The detailed procedure for an intermediate scale of 173.8 mmols is as follows. In a 500 mL reaction flask t-BuOH (150 mL), water (150 mL), benzyl bromide (173.8 mmol) and sodium azide (192.8 mmol) were added and mixed. The resulting blend was stirred at room temperature for 24 h. Thereafter, phenylacetylene (173.8 mmol), ascorbic acid (34.8 mmol), sodium hydroxide (35.0 mmol) and pentahydrated copper sulfate (17.4 mmol) were added sequentially and in one portion. The final mixture was stirred at room temperature for an additional time of 24 h.

Once past this time, the whitish solids were separated by filtration under vacuum, washed with copious amounts of distilled water and air dried to obtain the desired product as a grayish white powder in 95% yield. The NMR analysis showed that the product was obtained with analytical purity. H-NMR (400 MHz, CDCl₃): δ 5.57 (s, 2H), 7.29-7.33 (m, 3H), 7.36-7.42 (m, 5H), 7.66 (s, 1H), 7.79-7.81 (m, 2H) ppm; C-NMR (100 MHz, CDCl₃) δ: 54.4, 119.6, 125.8, 128.2, 128.3, 128.90, 128.92, 129.3, 130.6, 134.8, 148.4 ppm; IR (KBrpellets): δ 3141, 1469, 1451, 1362, 1224, 1073, 1046, 768, 732, 697 cm⁻¹. Melting point: 124-125° C. These data agree with previously published literature [29, 37].

### 2.2 Electrochemical Testing

The steel cylinder working electrode (WE) were cross-sectioned and encased in epoxy. The surface was polished using 120 grit to 600 grit SiC paper, resulting in an exposed area of 0.499±0.01 cm². A Pt mesh was used as the counter electrode (CE). In terms of the reference electrodes (RE), a Saturated Calomel Electrode (SCE) was used for solutions with chloride and a Mercury Mercurous Sulfate Electrode (MSE) for solutions without chloride, satisfying the electrochemical cell requirements of ASTM standard G5 [38]. Though the experiments without chlorides were measured with MSE, the results were converted to SCE.

The WE, RE, and CE were placed in a test cell containing a synthetic pore solution (SPS). This SPS mimics the concrete pore environment and followed a formula consisting of 8.33 g/L NaOH+23.3 g/L KOH+2.0 g/L Ca(OH)₂ (pH:13.6) [33-35, 39]. The SPS was used as a base for the electrolyte solutions presented in Table 2. These solutions were prepared using deionized water from a Milli-Q Water Purification System (resistivity: 18.2-18.7 MΩ·cm) and analytically pure grade reagents.

Initial tests were run to find the stability/solubility of BPT in SPS. From these, the BPT was shown to be stable in the alkaline solution, but it was noted that the compound in its current form was unable to dissolve in water, and a co-dissolver was needed. Several co-dissolvers were tested (ethyl alcohol 95%, isopropyl alcohol, acetone, dimethylsulfoxide, dimethylformamide, ethylenglycol, diethylenglycol, and 1-Methyl-2-pyrrolidinone), to aid in the solubility of the BPT in SPS. From these co-dissolvers, the organic compound N-methyl-2-pyrrolidone (NMP) performed the best and was selected at a concentration of 2.35 mg of BPT/mL of NMP (BPT saturated NMP solution).

TABLE 2
Chemical composition and pH of solutions with and
without 2M NaCl used for electrochemical testing.

| | NaOH | KOH | Ca(OH)₂ | NMP | BPT | pH | pH |
|---|---|---|---|---|---|---|---|
| Electrolyte | (g/L) | (g/L) | (g/L) | (mL/L) | (M) | (w/o Cl) | (w/Cl) |
| SPS | | | | — | — | 13.6 | 13.3 |
| 100 mL NMP | | | | 100.12 | — | 13.1 | 12.8 |
| 400 mL NMP | | | | 400.48 | | 14.2 | 13.6 |
| 1 mM BPT + NMP | 8.33 | 23.3 | 2.0 | 100.12 | 0.2353 | 13.7 | 13.4 |
| 2 mM BPT + NMP | | | | 200.24 | 0.4706 | — | — |
| 3 mM BPT + NMP | | | | 300.36 | 0.7059 | 13.7 | 13.2 |
| 4 mM BPT + NMP | | | | 400.48 | 0.9411 | 14.1 | 13.6 |

The testing procedure involved an initial pH measurement of the solution followed by purging the cell with CO₂-free air (300 cc/min or more) for 5 minutes, prior to introducing the WE and throughout the experiment to reduce the effects of carbonation. More details can be found elsewhere [40]. 2 M NaCl was chosen for the experiments since previously tested concentrations [40] were not able to break the passive film generated in this highly alkaline environment. After chloride addition, the electrolyte was stirred for 4 h, while continuing to purge with CO₂-free air. Then stirring stopped and purging was continued for 20 h to allow for passivation of the substrate. If experiments did not include chlorides, the test cell continued to purge for 24 h after the initial 24 h of purging were completed. This procedure is similar to ASTM G180 [41]. The difference is related to the use of SPS instead of a cement slurry. Using a Gamry Reference 600+Potentiostat, different electrochemical tests were performed including open circuit potential (OCP), linear polarization resistance (LPR) and Anodic Cyclic Polarization (CYP).

LPR tests were performed from −0.030 V to +0.030 V from OCP, with a scan rate of 0.167 mV/s, in accordance with ASTM G59. CYP ran at a scan rate of 0.167 mV/s, beginning at 0.030 V more negative than the OCP with a maximum i of 1 mA/cm². Tests were performed to verify that pitting was occurring and not crevice corrosion occurred during CYP. After each experiment, the specimen was rinsed with D.I. water, degreased with ethanol, and dried. Surface images were taken using BX53 Olympus microscope to evaluate the corrosion morphology. Each of the conditions presented in Table 2 were tested at least 3 times, with and without chlorides.

### 2.3 BPT Film Characterization by XRD

Four 3 mm height discs were cut from a/4 in diameter steel rebar and one of their flat faces was polished using silicon carbide sandpaper with 250, 400 and, 600 grit. Subsequently each disc was immersed for 90 seconds in 10 mL of a freshly prepared Clarke solution [42], rinsed thoroughly with water, acetone and dried under a soft stream of dry N₂. The crystalline structure of the samples was analyzed on their sheeny face employing X-Ray powder diffraction (XRD) using a Bruker D8 Advance Eco diffractometer applying Cu-Kα_{1,2} radiation. All samples were scanned at 40 kV and 25 mA in the 20 range of 31-66° and 2.0 s of step time per 2θ=0.019°. The crystalline planes used to perform the quantitative phase analysis (QPA) are [1,1,0] for iron at 2θ=44.6°; [2,0,0] for lepidocrocite at 2θ=47.4° and, [2,2,0] for goethite at 2θ=33.6°.

### 3. Results and Discussion

### 3.1 BPT Synthesis

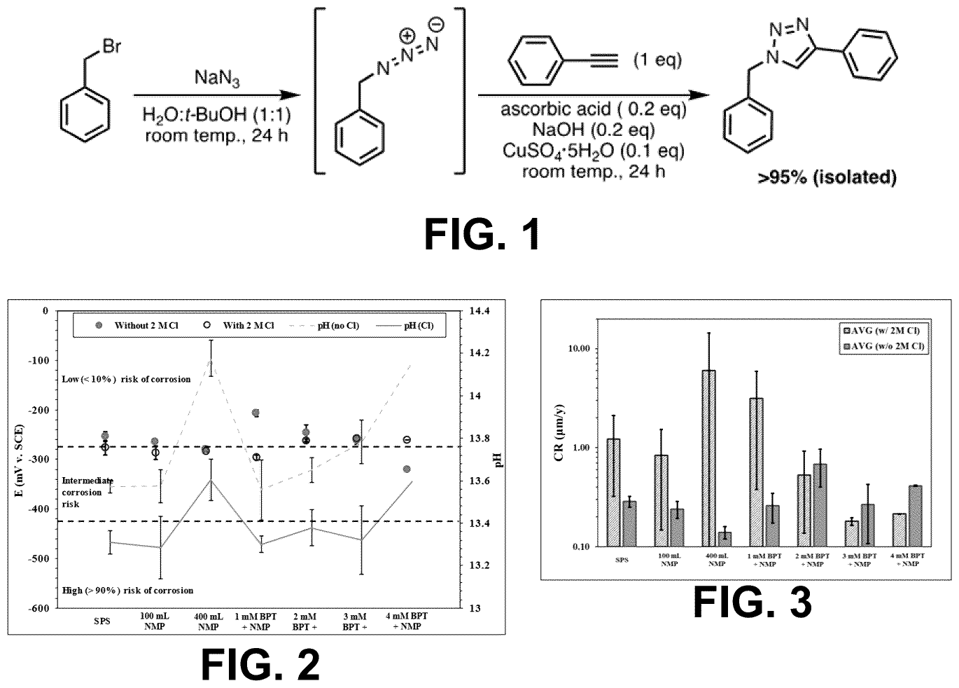

BPT is a nitrogenated heterocyclic compound that has been prepared using different chemical strategies. Just to name a few, we can name the Huisgen Azide-Alkyne 1,3-dipolar cycloaddition [43], the Ru-catalyzed Azide-Alkyne cycloaddition (RuAAC) [44], the copper-catalyzed decarboxylative cycloaddition of propiolic acids with organic azides [45] and the Nickel catalyzed Alkyne-Azide coupling (NiAAC) reaction [46]. However, in the nearly more than 60 different chemical syntheses reported in 2021 and the 25 synthetic methods reported so far in the first semester of 2022, the most applied strategy involves the CuAAC (copper-catalyzed Azide-Alkyne coupling) reaction. These recently reported synthetic strategies involve the use of state-of-the-art catalytic systems like Cu-based metal-organic frameworks (MOFs) [47-50], copper coordination polymers [51], polymer-supported catalysts [52, 53], Cu coordination complexes [54-60] and, magnetic nanoparticles [61-66]. Nonetheless, despite the myriad of different catalytic systems employed, it seems that when several grams of 1,2,3-triazoles are needed, the use of the Sharpless-Fokin catalyst (CuSO₄/sodium ascorbate) [67] is by far the most logical choice regardless of the sub-stoichiometrical amount of catalyst needed (10 mol %). For this work, it was decided to apply a previously reported synthetic method for the preparation of BPT [29] albeit, some modifications were made to improve its greenness. Thus, to avoid the use of toxic solvents such as the one employed in the former synthesis (THF), to dodge the excessive handling of chemicals and reduce the production of chemical waste, the preparation of the inhibitor was performed using a one-pot synthesis consisting of two sequential chemical reactions (FIG. 1).

As the first part of the synthesis involves a bimolecular nucleophilic substitution reaction taking place over a very reactive substrate (benzyl bromide), it was decided to use the solvent system reported by Folkin [67] for the whole process. This solvent mixture has a relative permittivity (εₒ) of 17.4 [68] that classifies it as a less polar system than water but a little more polar than THF. According to the Hughes-Ingold rules [69], this slight increase in the polarity of the system (from THF to the H₂O:t-BuOH mixture) should not have a high impact on the reaction rate, but to assure reaction completion, the system was stirred at room temperature for 24 h. After this, a simple sequential addition of the remaining reagents followed by a reaction time of 24 h at room temperature resulted on the desired compound with analytical purity and an isolated yield of 97.5%. This methodology not only avoided the isolation and handling of the potentially explosive benzyl azide, but allowed us to scale up from 10 mmol to 1 mol and obtain the final product in yields higher than 95%.

### 3.2 Electrochemical Testing

### OCP and LPR

The OCP and pH measurements of the different conditions are presented in FIG. 2. Applying the criteria from ASTM C876 [70], it can be seen that most conditions are at low risk of corrosion. Systems measuring more positive than −276 mV vs. SCE have a tendency to be passive and systems measuring more negative than −276 mV vs. SCE have a tendency to be actively corroding [70, 71]. The corrosion morphology of the samples was observed after testing to rule out crevice corrosion.

Despite the reduction of OH⁻ concentration in the solutions containing NMP, the pH increased with the inclusion of NMP. This co-dissolver has a lower pH (11) than the SPS (13.6). When 400 mL of NMP was added, a decrease in pH was expected. But it was not the case, the pH increased to 14.2. In addition, an increase in temperature and water evaporation was observed, which indicated that some exothermic reactions took place. This may be the cause of the decrease in OCP, as well as some variety in solution pH, but more investigation is needed. Nevertheless, this is not within the scope of this investigation.

Once the BPT was added to the mix, the OCP value initially increased before decreasing to the lowest value out of all the conditions, even though NMP was present. This effect is significant when chlorides are added to the mix, where the OCP becomes more noble with increasing BPT concentration, indicating a tendency to enhance corrosion inhibition in presence of chloride ions. OCP is only a method that provides the electrochemical steady state tendency of the metal in its environment to corrode. Therefore, other electrochemical techniques were needed to provide more information about the corrosion kinetics.

The results of the LPR measurements for the different conditions tested are presented in FIG. 3 as CR, which was determined using an estimated Stem-Geary coefficient (B) of 26 mV (corroding bars) and Rp values calculated, all following ASTM G59 [72, 73]. Using the previously mentioned criteria, the results showed that, except for 1 mM BPT, the rebar remains passive in most of the conditions evaluated that contain BPT and NMP (Rp>250000 Ω·cm², CR<1.16 μm/year and i_{corr}<0.1 μm/cm²) [74]. The Rp values increased with the addition of BPT in Cl⁻ containing SPS, and in turn, reduced the CR observed for the carbon steel rebar samples. These CR values decreased with higher BPT concentrations, with the lowest value achieved at a BPT concentration of 3 mM and having a slight increase when the concentration is increased to 4 mM. Based on these results, the optimal concentration of BPT to inhibit corrosion on carbon steel rebar was 3 mM.

Low values of CR were also observed for the condition that contains only NMP at its highest concentration (400 mL NMP) without the addition of Cl⁻. In this case, the decrease in CR was the result of an increase in pH for the conditions containing only NMP (pH of 14.2 vs 13.6). At high pH values, the passive film increases in strength [1, 75]. The organic nature of the NMP could lead to absorption of this chemical to the substrate and therefore compete with BPT for the active sites on the WE. Nevertheless, in presence of 2 M NaCl, the NMP (only) related passive film was not strong enough to inhibit the corrosion rate of steel in SPS.

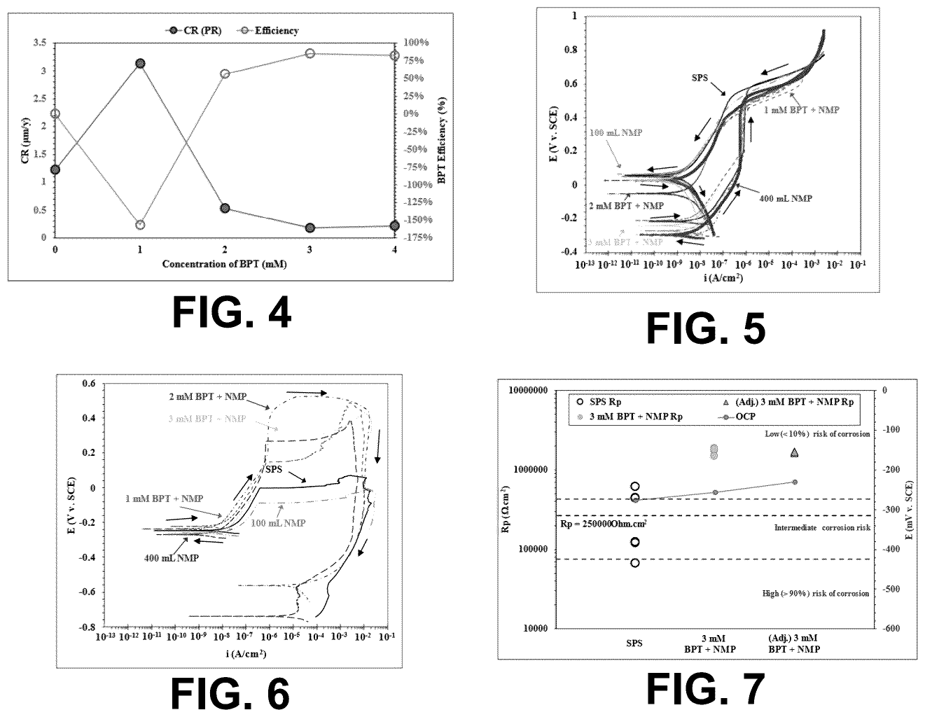

The efficiency (IE, %) of the BPT was calculated using the CR results when Cl⁻ was added to the control solution (CR_{SPS}) and solutions containing BPT (CR_{BPT}) [8], using Equation (1). The results were compared to the CR in FIG. 4. It can be observed that the BPT efficiency increases as the BPT concentration increases after a BPT concentration of 2 mM, with the highest efficiency (85.2%) obtained for 3 mM BPT. As no improvement of corrosion inhibition was noted with increasing the concentration of BPT higher than 3 mM, the 4 mM BPT+NMP condition was not further studied.

$$IE = \frac{{CR}_{SPS} - {CR}_{BPT}}{{CR}_{SPS}} ( x 100 ) \qquad ( 1 )$$

In summary, BPT addition in sufficient amount results in the formation of a protective outer layer that is integrated in the passive layer naturally formed (high pH conditions).

Given the organic nature of BPT, the compound could be classified as a filmic OCI [29]. OCIs generally inhibit corrosion by chemisorption, physisorption, or both mechanisms by using a polar group which can have nitrogen, oxygen, and/or sulfur atoms with multiple bonds, onto the surface of the metal, then promote growth and maintenance of the intrinsic passive film [8, 21, 22]. BPT contains a polar group of nitrogen and is suspected to be responsible for the adsorption onto the rebar surface which enhances the passive film already provided by the alkaline concrete environment. This passive film can be considered as a physical barrier composed of an enhanced passive film containing organic compounds related to the inhibitor.

### CYP

The results of the CYP tests are presented in FIGS. 5 and 6 for conditions without Cl⁻ and with Cl⁻ additions, respectively. The results in FIG. 5 indicate that the rebar is spontaneously passive for the conditions tested. Furthermore, the scans revert at current densities lower than the values obtained during the upward scan. This indicates a negative hysteresis, which represents that the integrity of the passive films is maintained during the test. The sudden increase in current density taking place at higher potentials in the upward scan is related to the oxygen evolution reaction (OER). This behavior in the absence of aggressive ions such as Cl⁻ has been reported previously [76]. The transpassive potential (Eₜᵣₐₙₛ) values obtained (average of 0.535 V vs. SCE) are above the Nernst potential for OER for the range of pH measured (pH: 13.6-14.2; E_{O2}: 0.186-0.150 V vs. SCE). It can be seen in the figure that conditions not containing BPT resulted in higher Eₜᵣₐₙₛ values than conditions containing BPT. Despite the BPT containing solutions having similar pH values to solutions not containing BPT, they had lower Eₜᵣₐₙₛ values and were more comparable to those of the solution with the highest pH (400 mL NMP; pH: 14.2).

FIG. 6 shows the effect of 2 M NaCl in the behavior of the passive film with/without BPT inhibitor. It is observed that for all conditions evaluated the passive film on the steel was formed, but the pitting potential (Eₚ) changed, being higher with the increase in BPT concentration (Table 3). These results show, again, that the BPT increases the resistance of the passive film on steel.

TABLE 3
OCP and Eₚ for carbon steel rebar in different SPS solutions
with BPT concentrations after exposure following passivation
of the WE in presence of 2M NaCl.

| Condition | OCP (mV vs. SCE) | Eₚ (mV vs. SCE) |
|---|---|---|
| SPS | −246.19 | −1.12 |
| 100 mL NMP | −251.99 | −87.38 |
| 400 mL NMP | −268.53 | 267.48 |
| 1 mM BPT | −219.58 | 149.25 |
| 2 mM BPT | −236.13 | 404.04 |
| 3 mM BPT | −258.55 | 302.06 |

In presence of chlorides, when the BPT is added to the control SPS, the Eₚ values are considerably higher, showing its effectiveness as a CI. These results suggest that the BPT successfully adsorbs onto the rebar surface and enhances the protective passive film. Despite the reduction of the hydroxide concentration in the solutions prepared as the BPT concentration was increased (the reduction in hydroxide concentration alone makes it more difficult for the formed oxide to protect the underlying rebar at the same Cl-concentration), the Eₚ increased with BPT concentration. The order in Eₚ from higher to lower values followed, 2 mM BPT+NMP>3 mM BPT+NMP>1 mM BPT+NMP.

The 400 mL+NMP condition also produced a high E_{P} in presence of Cl⁻. As was indicated before, this is most likely due to the high pH value of the electrolyte, which increases the Cl⁻ concentration needed to break the passive film. These higher values of E_{P} show better resistance to corrosion, but as it can be seen in FIG. 6, no condition in presence of 2 M NaCl was able to repassivate and complete the hysteresis loop to obtain a repassivation potential (E_{R}) above OCP. This occurred because the pits that were created during the tests were made in a highly aggressive environment (2 M NaCl), creating deeper pits than under lower Cl⁻ concentration, making it more difficult for the pits to repassivate. This phenomenon has been noted in previous testing in SPS [39]. This observation highlights the strength the BPT has to inhibit corrosion in even highly aggressive Cl⁻ environment.

BPT Effect after Hydroxide Adjustment

The solutions tested previously (Table 2) presented a decrease in OH⁻ concentration as the BPT concentration and NMP amount increased (60% to 90% of the concentration for SPS), making the environment less prone to passivate the rebar and creating a more corrosive environment. Therefore, another solution was prepared which kept the same hydroxide concentration as the SPS (Table 4). The optimal concentration (3 mmol/L of BPT+NMP) was chosen for this solution and the same test was performed.

Slight improvements were observed from the pH values presented in Table 2 (3 mM BPT+NMP: 13.7 without 2 M Cl⁻ and 13.2 with 2 M Cl⁻) to the pH values presented in Table 4 (Adj. 3 mM BPT+NMP: 13.9 without 2 M NaCl and 13.4 with 2 M NaCl), which is expected as a result from the increase in hydroxide concentration.

TABLE 4
Concentration of solution used to compare BPT effect
under same hydroxide concentration as SPS.

| | NaOH | KOH | Ca(OH)₂ | NMP | BPT | pH | pH |
|---|---|---|---|---|---|---|---|
| Condition | (g/L) | (g/L) | (g/L) | (mL/L) | (mM) | (No Cl⁻) | (w/Cl⁻) |
| Adj. 3 mM BPT + NMP | 8.33 | 23.3 | 2.0 | 300.36 | 3 | 13.9 | 13.4 |

The results of the OCP and LPR tests are compared in FIG. 7, using the old and new procedure to prepare the solution with BPT and the control (without BPT). The OCP shifted to higher values (more noble) in the new BPT solution (Adj.) when compared to the old one, while the Rp results were very similar to the old solution. The inhibitor efficiency was calculated using Equation (1) and a nominal increase of only 0.1% was found. CYP were also run to evaluate the adjustment of the pH in the SPS solution with BPT. The results were similar to LPR.

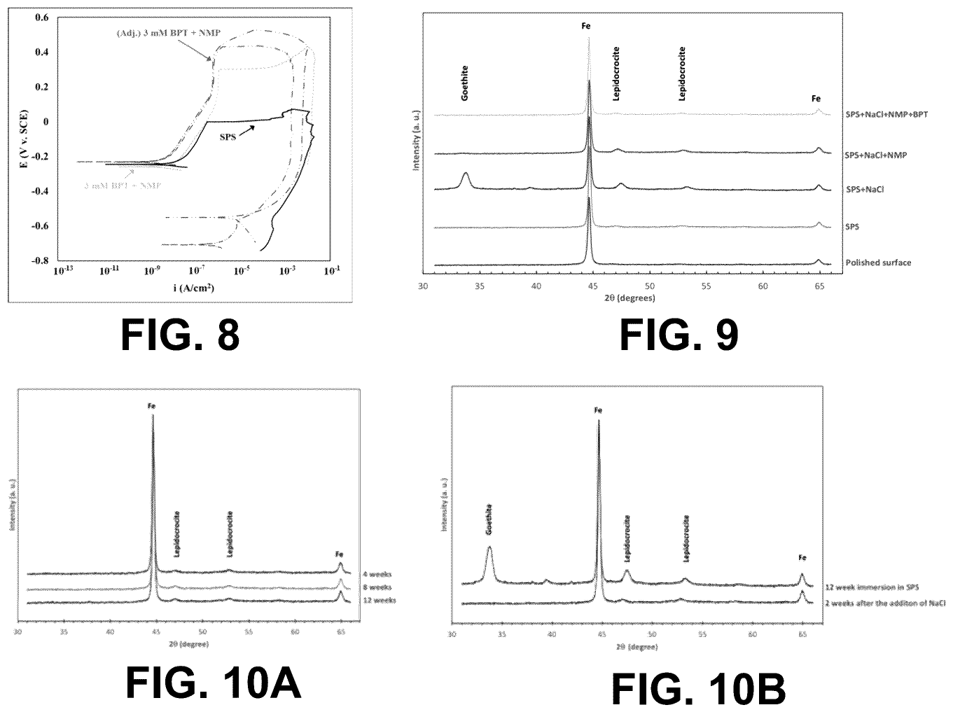

The results of the CYP tests are presented in FIG. 8. A positive hysteresis loop is present in the figure, indicating pitting because of the Cl⁻ ions. The effectiveness of the BPT can already be seen by the large difference in Eₚ values between the control SPS and the solutions containing the optimal concentrations determined (3 mM BPT+NMP and Adj. 3 mM BPT+NMP). Also, the Eₚ is higher in this new Adj. solution (429.53±1.29 mV vs. SCE) when compared to the other one. This could be due to the increased pH values the new solution has (from 13.7 to 13.9), as high pH values increase the strength of the passive film, increasing the amount of Cl⁻ needed to break it. Despite this increase in Eₚ values, this condition was again unable to repassivate and complete the hysteresis loop to produce a E_{R} above OCP, possibly due to the depths of the pits created in this highly aggressive environment (2 M NaCl) [39, 76].

### 3.3. Effect of BPT Presence in Steel Surface Film Chemistry (X-Ray Diffraction)

### Surface Characterization

The sheeny face of one of the discs was analyzed using X-Ray powder diffraction (XRD) and their diffractogram showed only the expected pattern for iron (FIG. 9, red curve). Subsequently, each one of the discs was immersed in a solution with the composition as described on Table 5 and their surfaces were analyzed using XRD four weeks later. The diffraction patterns are shown in FIG. 9 and the quantitative phase analysis (QPA) data is given on Table 6.

TABLE 5
Composition of the solutions used to
characterize the carbon steel surface

| Disc Number | SPS | 2M NaCl | NMP* | 3 mM BPT |
|---|---|---|---|---|
| 1 | ✓ | | | |
| 2 | ✓ | ✓ | | |
| 3 | ✓ | ✓ | ✓ | |
| 4 | ✓ | ✓ | ✓ | ✓ |

*Volume of co-solvent used was the minimal required to solubilize BPT.

TABLE 6
XRD quantitative phase analysis of carbon steel after a four-
week immersion in the solutions described in Table 9.

| | Area under | Area under | Area under | | |
|---|---|---|---|---|---|
| Disc | main Fe | main Lepido- | main Goethite | A_{Fe}/ | A_{Fe}/ |
| Number | signal | crocite signal | signal | A_{Lep} | A_{Goe} |
| 1 | 34.096 | 1.256 | — | 27,146 | — |
| 2 | 31.628 | 3.413 | 17.362 | 9,270 | 1,822 |
| 3 | 31.753 | 2.598 | — | 12,222 | — |
| 4 | 33.501 | 1.245 | — | 26,908 | — |

After one month, disc 1 showed only a trace amount of Lepidocrocite (g-FeOOH). The small bumps at approximately 47 and 53° in FIG. 8 (green line) and the high A_{Fe}/A_{Lep} ratio obtained by the QPA (Table 6) confirms that the formation of this oxide occurred at a very low extent. On its part, the XRD pattern of disc 2 showed the presence of Lepidocrocite and of α-FeO(OH) (Goethite) (FIG. 9, blue line). The QPA for this sample confirmed that the amount of oxidation products formed after one month, is not negligible. For disc 3, it is noteworthy to mention that this sample got corroded to a lesser extent than disc 2. The XRD patterns of these two samples differ not only in the intensity of the Lepidocrocite signals (FIG. 9, blue and purple lines) but also the QPA for disc 3 shows a higher A_{Fe}/A_{Lep} ratio (Table 6). Furthermore, it also seems that NMP inhibits the formation of Goethite as this signal is absent in the diffraction patterns of discs 3 and 4. This result agree with the electrochemical characterizations, as they provided some evidence that this compound has some corrosion inhibition properties. Finally, and as expected, the specimen that showed the best corrosion resistance was disc 4. The diffraction pattern of this sample shows little evidence of oxidation as the signals for Lepidocrocite are almost imperceptible (FIG. 9) and the A_{Fe}/A_{Lep} ratio is similar to the one obtained for the passivated disc 1.

To have an insight about the depassivation action of chloride ions, three freshly polished rebar discs were immersed on SPS and their surface was analyzed by XRD after 4, 8 and 12 weeks. As it can be seen on FIG. 10a, the shape of the XRD patterns do not show any significant changes over time. However, this steady state is perturbed when the samples get in contact with Cl⁻ ions. As shown in FIG. 10b, oxidation species begin to appear whenever NaCl is added to the solution. Two weeks after the addition of chloride ions to a sample that was kept in SPS for 12 weeks, showed that corrosion took place at a visible extent. The XRD pattern of this sample showed the appearance of a defined signal at 33.6° and an intensity increase for the signal at 47.4°. The first signal can be related to Geothite and the second signal confirms the formation of Lepidocrocite [77]. Both Goethite [α-FeO(OH)] and Lepidocrocite (g-FeOOH) have been reported as products of the corrosion of iron in alkaline media [78, 79].

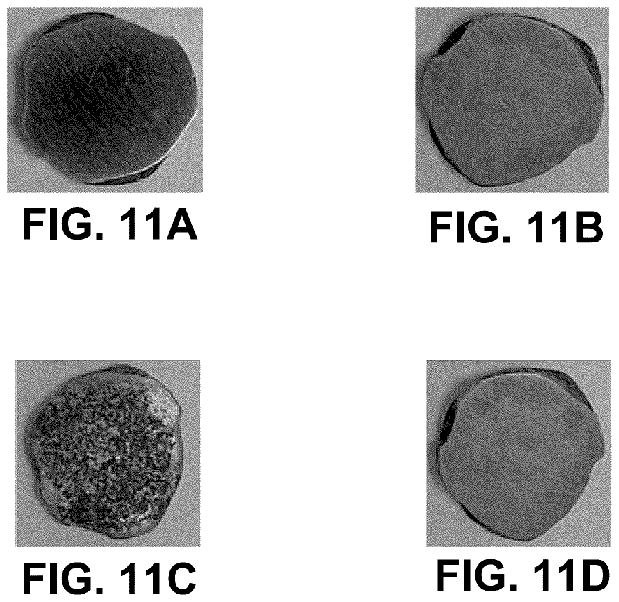

Finally, the corrosion morphology after exposure is shown in FIG. 11 where it is evident that the initial freshly polished shiny surface of disc 1 (FIG. 11a) changes its appearance after being immersed for a week in SPS solution (FIG. 11b) and after the addition of solid NaCl to the original solution (FIG. 11c). Furthermore, after a month immersion in SPS in the presence of Cl⁻ ions, NMP and BPT, disc 4 (FIG. 11d) has a physical appearance that is similar to disc 1 (FIG. 11b). This similarity, that is also confirmed by their diffraction patterns and their quantitative phase analysis (FIG. 11 and Table 6) give us enough evidence of the effective corrosion inhibition behavior of the BPT molecule.

To this point, BPT has given results that confirm its potential use as a GCI for reinforced concrete. The collected evidence that supports the hypothesis of a film formation that shields the metallic surface from the corrosive attack of Cl⁻. Considering the structure of BPT, it can be expected that the main inhibition mechanism is similar to the one described by Tang et al. [80] for (1-benzyl-1H-1,2,3-triazol-4-yl)methanol (BTM), a triazole containing molecule that like BPT, has also been reported to inhibit mild steel corrosion in acidic media. In this paper, the authors conclude from DFT calculations, that BTM absorbs on the metal surface either through electron donation of the 71-bond electrons of the benzyl segment and by the acceptance of electrons by the triazole moiety. As BPT has a benzyl and a triazole segment on its structure, a similar absorption behavior might be foreseen.

### 4. Conclusions

In the present Example the use of BPT as a GCI for steel was evaluated in SPS solutions using a carbonation free and rebar passivation stabilization protocol prior Cl⁻ addition, and a combination of experimental techniques, including OCP, LPR, CYP, EIS, and XRD. From this work the following conclusions were obtained:
BPT was synthesized with a high yield using a greener chemical strategy. The synthesis was improved by the reduction of both, solvents and the handling of dangerous chemicals. All these, following the 12 principles of green chemistry to produce a low-cost, eco-friendly GCI.
A co-dissolver like NMP must be used with the BPT to perform test in aqueous solutions.
BPT can inhibit the corrosion of carbon steel rebar in SPS with 2 M NaCl at concentrations higher than 2 mM. But, the optimal concentration was found to be 3 mM BPT with an efficiency of 85.2%.
XRD and corrosion morphology analysis demonstrated the reduction of oxides content on the rebar surface film in presence of BPT in SPS with NaCl (from A_{Fe}/A_{Lep}=9,270 to 26,908 and from A_{Fe}/A_{Goe}=1,822 to 0), further providing evidence that BPT can inhibit corrosion in SPS.

### REFERENCES

1. Gagg, C. R., Cement and concrete as an engineering material: An historic appraisal and case study analysis. Engineering Failure Analysis, 2014. 40: p. 114-140.
2. Bertolini, L., et al., Corrosion of Steel in Concrete: Prevention, Diagnosis, Repair. 2014, Weinheim: John Wiley & Sons, Incorporated.
3. Graves, H., et al., Expanded Materials Degradation Assessment (EMDA) Volume 4: Aging of Concrete and Civil Structures, O.o.N.R. Research, Editor. 2014, United States Nuclear Regulatory Commission. p. 137.
4. ACI, Protection of Metals in Concrete Against Corrosion. 2001: American Concrete Institute.
5. Goñi, S. and C. Andrade, Synthetic concrete pore solution chemistry and rebar corrosion rate in the presence of chlorides. Cement and concrete research, 1990. 20(4): p. 525-539.
6. Volpi, E., et al., Electrochemical characterization of mild steel in alkaline solutions simulating concrete environment. Journal of Electroanalytical Chemistry, 2015. 736: p. 38-46.
7. Yu, H., K. T. K. Chiang, and L. Yang, Threshold chloride level and characteristics of reinforcement corrosion initiation in simulated concrete pore solutions. Construction & building materials, 2012. 26(1): p. 723-729.
8. Rivetti, M. L. S., et al., Corrosion Inhibitors for Reinforced Concrete, in Corrosion Inhibitors, Principles and Recent Applications. 2018.
9. Ress, J., et al., pH-Triggered Release of NaNO₂ Corrosion Inhibitors from Novel Colophony Microcapsules in Simulated Concrete Pore Solution. ACS Appl. Mater. Interfaces, 2020. 12: p. 46686-46700.
10. Mammoliti, L., C. M. Hansson, and B. B. Hope, Corrosion inhibitors in concrete Part-II: Effect on chloride threshold values for corrosion of steel in synthetic pore solutions. Cement and concrete research, 1999. 29(10): p. 1583-1589.
11. Berke, N. S., et al., Corrosion of Steel in Cracked Concrete. Corrosion Journal, 1993. 49(11): p. 934-943.
12. Troconis de Rincón, O., et al., Long-term performance of ZnO as a rebar corrosion inhibitor. Cement & concrete composites, 2002. 24(1): p. 79-87.
13. Berke, N. S. and M. C. Hicks, Predicting long-term durability of steel reinforced concrete with calcium nitrite corrosion inhibitor. Cement and Concrete Composites, 2004. 26(3): p. 191-198.
14. Królikowski, A. and J. Kuziak, Impedance study on calcium nitrite as a penetrating corrosion inhibitor for steel in concrete. Electrochimica Acta, 2011. 56(23): p. 7845-7853.
15. Ramirez, E., E. Otero, and J. A. González, Determination of critical inhibition threshold in Fe Ca(OH)2 Cl—NO2-system. British Corrosion Journal, 1993. 28: p. 121-124.
16. Rosemberg, A. M., J. M. Gaydis, and e. al, A Corrosion Inhibitor For-mu-lated with Calcium Nitrite for Used in Reinforced Concrete. ASTM-STP, 1977. 629.
17. Mohamed, A., D. P. V. Jr., and D. M. Bastidas, Effect of cations on the activity coefficient of NO₂⁻/NO₃⁻ corrosion inhibitors in simulated concrete pore solution: An electrochemical thermodynamics study. Corrosion Science, 2022. 206.
18. Sangoju, B., et al., Influence of PCE-SP and calcium nitrite inhibitor on mechanical and durability parameters of concrete. Journal of Scientific & Industrial Research, 2015. 74: p. 82-87.
19. Lee, H. S., et al., Corrosion Inhibitors for Reinforced Concrete: A Review, in Corrosion Inhibitors, Principles and Recent Applications. 2018.
20. Mohamed, A., D. P. Visco, and D. M. Bastidas, Significance of π-Electrons in the Design of Corrosion Inhibitors for Carbon Steel in Simulated Concrete Pore Solution. Corrosion, 2021. 77(9): p. 976-990.
21. Jones, D. A., Principles and Prevention of Corrosion. 2nd ed. 1996, Upper Saddle River, NJ 07458: Prentice Hall.
22. Ormellese, M., et al., A study of organic substances as inhibitors for chloride-induced corrosion in concrete. Corrosion Science, 2009. 51(12): p. 2959-2968.
23. Kim, C., et al., Electrochemical evaluation of epoxy-coated-rebar containing pH-responsive nanocapsules in simulated carbonated concrete pore solution. Progress in Organic Coatings, 2021. 161.
24. Pereira, E. V., et al., Long-Term Efficiency of Two Organic Corrosion Inhibitors for Reinforced Concrete. Materials Science Forum, 2010. 636-637: p. 1059-1064.
25. Shehata, O. S., L. A. Korshed, and A. Attia, Green Corrosion Inhibitors, Past, Present, and Future, in Corrosion Inhibitors, Principles and Recent Applications. 2018.
26. Jang, J. K., Amines as occupational hazards for visual disturbance. Industrial health, 2016. 54(2): p. 101-115.
27. Abdulrahman, A. S. and I. Mohammad, Green Plant Extract as a passivation-promoting Inhibitor for Reinforced Concrete. International Journal of Engineering Science and Technology (IJEST), 2011. 3(8): p. 7.
28. Asipita, S. A., et al., Green Bambusa Arundinacea leaves extract as a sustainable corrosion inhibitor in steel reinforced concrete. Journal of Cleaner Production, 2014. 67: p. 139-146.
29. Fernandes, C. M., et al., Green synthesis of 1-benzyl-4-phenyl-1H-1,2,3-triazole, its application as corrosion inhibitor for mild steel in acidic medium and new approach of classical electrochemical analyses. Corrosion science, 2019. 149: p. 185-194.
30. Mennucci, M. M., et al., Evaluation of benzotriazole as corrosion inhibitor for carbon steel in simulated pore solution. Cement and Concrete Composites, 2009. 31(6): p. 418-424.
31. Klodian Xhanari, M. F., Organic corrosion inhibitors for aluminum and its alloys in chloride and alkaline solutions: A review. Arabian Journal of Chemistry, 2019. 12(8): p. 4646-4663.
32. Duffó, G. S. and S. B. Farina, Electrochemical behaviour of steel in mortar and in simulated pore solutions: Analogies and differences. Cement and Concrete Research, 2016. 88: p. 211-216.
33. Li, L. and A. A. Sagüés, Chloride corrosion threshold of reinforcing steel in alkaline solutions: Cyclic polarization behavior. Corrosion (Houston, Tex.), 2002. 58(4): p. 305-316.
34. Li, L. and A. A. Sagüés, Chloride corrosion threshold of reinforcing steel in alkaline solutions: Effect of specimen size. Corrosion (Houston, Tex.), 2004. 60(2): p. 195-202.
35. Li, L. and A. A. Sagüés, Chloride corrosion threshold of reinforcing steel in alkaline solutions: Open-circuit immersion tests. Corrosion (Houston, Tex.), 2001. 57(1): p. 19-28.
36. Anastas, P. T. and J. C. Warner, Green Chemistry: Theory and Practice. Oxford University Press, 1998.
37. Sharghi, H., R. Khalifch, and M. M. Doroodmand, Adv. Synth. Catal., 2009. 351: p. 207-218.
38. ASTM, Standard Reference Test Method for Making Potentiodynamic Anodic Polarization Measurements (G5-14). 2018: www.ASTM.org.
39. Li, L. and A. A. Sagues, Effect of Chloride Concentration of the Pitting and Repassivation Potentials of Reinforcing Steel in Alkaline Solutions, in Corrosion 99. 1999, NACE International.
40. Dacio, L. J. P., EVALUATION OF 1-BENZYL-4-PHENYL-1H-1,2,3-TRIAZOLE AS A 40. CORROSION INHIBITOR IN REINFORCED CONCRETE ELEMENTS, in Department of Civil and Environmental Engineering. 2021, THE UNIVERSITY OF TEXAS AT SAN ANTONIO: ProQuest. p. 155.
41. ASTM, Standard Test Method for Corrosion Inhibiting Admixtures for Steel in Concrete by Polarization Resistance in Cementitous Slurries (G180-13). 2013 (2019): www.ASTM.org.
42. ASTM, Standard Practice for Preparing. Cleaning. and Evaluating Corrosion Test Specimen (G1-03). 2003 (2017): www.ASTM.org.
43. Rostovtsev, V. V., et al., A Stepwise Huisgen Cycloaddition Process: Copper(I)-Catalyzed Regioselective “Ligation” of Azides and Terminal Alkynes. Angewandte Chemie (International ed.), 2002. 41(14): p. 2596-2599.
44. B C, B., et al., Ruthenium-catalyzed azide-alkyne cycloaddition: scope and mechanism. J Am Chem Soc, 2008. 138(28): p. 8923-30. Erratum in: J Am Chem Soc. 2008 Nov. 5; 130(44):14900.
45. Wang, X. X., et al., Copper-Catalyzed Decarboxylative Cycloaddition of Propiolic Acids. Azides. and Arylboronic Acids: Construction of Fully Substituted 1,2,3-Triazoles. The Journal of Organic Chemistry, 2020. 85 (5): p. 3576-3586.
46. Camberlein, V., et al., Multi-component reaction for the preparation of 1.5-disubstituted 1,2,3-triazoles by in-situ generation of azides and nickel-catalyzed azide-alkyne cycloaddition. Tetrahedron Letters, 2021. 73.
47. Fan, Z., et al., Defect engineering: an effective tool for enhancing the catalytic performance of copper-MOFs for the click reaction and the A3 coupling. Catalysis Science & Technology, 2021. 11(7): p. 2396-2402.
48. Yamane, I., et al., Ultrahigh-pressure preparation and catalytic activity of MOF-derived Cu nanoparticles. Nanomaterials, 2021. 11(4).
49. Wang, Z., et al., MOF-Derived Cu@N—C Catalyst for 1,3-Dipolar Cycloaddition Reaction. Nanomaterials, 2022. 12(7).
50. Kim, H., et al., Construction of Stable Metal-Organic Framework Platforms Embedding N-Heterocyclic Carbene Metal Complexes for Selective Catalysis. Inorganic Chemistry 2021. 60(24): p. 18687-18697.
51. Wang, F. F., et al., Functionalized resorcin[4]arene-based coordination polymers as heterogeneous catalysts for click reactions. New J. Chem., 2021. 45: p. 3181-3187.
52. Chetia, M., et al., Synthesis of copper containing polyaniline composites through interfacial polymerisation: An effective catalyst for Click reaction at room temperature. Journal of Molecular Structure 2021. 1233.
53. Deng, B., et al., Highly efficient catalytic performance on CuAAC reaction by polymer-like supramolecular self-assemblies-Cu(I) in aqueous solution. Applied Organometallic Chemistry, 2022. 36(6).
54. G. Mohammadnezhad, A. M. A., H. Görls, W. Plass, A. Sandleben, S. Schäfer, A. Klein., Redox Instability of Copper(II) Complexes of a Triazine-Based PNP Pincer. Eur. J. Inorg. Chem, 2021: p. 1140.
55. Khoshnoud, A. P., Ali Reza, Three-Component Synthesis of 1,4-Disubstituted 1,2,3-Triazoles using a Novel and Efficient Nano Alumina Based Cu(II) Catalyst. Organic Preparations and Procedures International, 2021. 53(6): p. 509-517.
56. Librando, I. L. M., Abdallah G.; Carabineiro, Sonia A. C.; da Silva, M. Fatima C. Guedes; Geraldes, Carlos F. G. C.; Pombeiro, Armando J. L., The catalytic activity of carbon-supported Cu(I)-phosphine complexes for the microwave-assisted synthesis of 1,2,3-triazoles. Catalysts, 2021. 11(2): p. 185.
57. Vilé, G. L., Jiaxu: Zhang, Zhenmei., Surface engineering of a Cu-based heterogeneous catalyst for efficient azide-alkyne click cycloaddition. Reaction Chemistry & Engineering 2021. 6(10): p. 1878-1883.
58. Gianvito Vilć, G. D. L., Sergio Tosoni, Alessandra Sivo, Vincenzo Ruta, Maarten Nachtegaal, Adam H. Clark, Stefano Agnoli, Yajun Zou, Aleksandr Savateev, Markus Antonietti, and Gianfranco Pacchioni., Azide-Alkyne Click Chemistry over a Heterogeneous Copper-Based Single-Atom Catalyst. ACS Catalysis, 2022. 12(5): p. 2947-2958.
59. Qiu, G. N., Paola; Martinez, Alexandre: Colomban, Cedric, A tris(benzyltriazolemethyl)amine-based cage as a CuAAC ligand tolerant to exogeneous bulky nucleophiles. Chemical Communications (Cambridge, United Kingdom), 2021. 57(18): p. 2281-2284.
60. Ma, L. G., Feifan; Ma, Jian-Fang, Two Cu(I)-based inorganic-organic complexes assembled with polyoxometalate and thiacalix[4]arene for efficient catalytic reactions. New Journal of Chemistry 2022. 46(15): p. 6995-7002.
61. Mirshafiee. S., Salamatmanesh, A, Heydari, A, A sustainable approach for efficient one-pot synthesis of 1-aryl 1,2,3-triazoles using copper iodide supported on 3-thionicotinyl-urea-modified magnetic nanoparticles in DES. Applied Organometallic Chemistry 2021. 35(3).
62. Rajabi-Moghaddam, H., Naimi-Jamal, M. R. & Tajbakhsh, M., Fabrication of copper(II)-coated magnetic core-shell nanoparticles Fe3O4@SiO2-2-aminobenzohydrazide and investigation of its catalytic application in the synthesis of 1,2,3-triazole compounds. Scientific Reports 2021. 11: p. 2073.
63. Godarzbod, F. M., Zohreh; Sacidian, Hamid; Rouhani, Morteza, Highly efficient synthesis of silica-coated magnetic nanoparticles modified with iminodiacetic acid applied to synthesis of 1,2,3-triazoles. Applied Organometallic Chemistry 2021. 35(3).
64. Khashei Siuki, H. G. K., Pouya; Bagherzade, Ghodsich., New Acetamidine Cu(II) Schiff base complex supported on magnetic nanoparticles pectin for the synthesis of triazoles using click chemistry. Scientific Reports 2022. 12(1): p. 3771.
65. Mohammadkhani, A. H., Akbar, Nano-magnetic Iron Oxide@Choline Acetate as a Heterogeneous Catalyst for the Synthesis of 1,2,3-Triazoles. Catalysis Letters 2022. 152(6): p. 1678-1691.
66. Pawar, A. G., Shivanand; Jagdale, Ashutosh; Patil, Sandip; Chandane, Wilson; Rashinkar, Gajanan; Patil, Suresh, Supported NHC-Benzimi@Cu Complex as a Magnetically Separable and Reusable Catalyst for the Multicomponent and Click Synthesis of 1,4-Disubstituted 1,2,3-Triazoles via Huisgen 1,3-Dipolar Cycloaddition. Catalysis Letters 2022. 152(6): p. 1854-1868.
67. Rostovtsev, V. V., et al., A Stepwise Huisgen Cycloaddition Process: Copper(I)-Catalyzed Regioselective “Ligation” of Azides and Terminal Alkynes. Angewandte Chemie International Edition, 2002. 41: p. 2596-2599.
68. Pius K. Kipkemboi, A. J. E., Solvent Polarity Studies of the Water+1-Butyl Alcohol and Water+t-Butylamine Binary Systems with the Solvatochromic Dyes Nile Red and Pyridinium-N-phenoxide Betaine. Refractometry and Permittivity Measurements. Aust. J. Chem., 1994. 47: p. 1771-1781.
69. Edward D. Hughes, C. K. I., Mechanism of substitution at a saturated carbon atom. Part IV. A discussion of constitutional and solvent effects on the mechanism, kinetics, velocity, and orientation of substitution. J. Chem. Soc., 1935: p. 244-255.
70. ASTM, Standard Test Method for Corrosion Potentials of Uncoated Reinforcing Steel in Concrete (C876-15). 2015: www.ASTM.org.
71. Elshami, A., et al., Effectiveness of corrosion inhibitors in simulated concrete pore solution. European journal of environmental and civil engineering, 2020. 24(13): p. 2130-2150.
72. ASTM, Standard Test Method for Conducting Potentiodynamic Polarization Resistance Measurements (G59-97). 1997 (2014): www.ASTM.org.
73. Andrade, C. and C. Alonso, Corrosion Rate Monitoring in the Laboratory and On-Site. Const. Build Mater., 1996. 10 (5): p. 315-328.
74. Rincon, O. T. d., et al., Manual for Inspecting. Evaluating and Diagnosing Corrosion in Reinforced Concrete Structures. Vol. 1st Edition in English. 2000, Maracaibo, Venezuela: CYTED.
75. Casey, H., K. Sen, and S. Ming. A New Method of Evaluating Corrosion-inhibiting Admixtures. in NACE International. 2019. Houston: NACE International.
76. Esmailzadeh, S., M. Aliofkhazraei, and H. Sarlak, Interpretation of Cyclic Potentiodynamic Polarization Test Results for Study of Corrosion Behavior of Metals: A Review. Protection of Metals and Physical Chemistry of Surfaces, 2018. 54(5): p. 976-989.
77. Faivre, D., Iron Oxides: From Nature to Applications. Weinheim: Wiley-VCH, 2016.
78. H. S. Wong, Y. X. Z., A. R. Karimi, N. R. Buenfeld, W. L. Jin., On the penetration of corrosion products from reinforcing steel into concrete due to chloride-induced corrosion. Corrosion Science, 2010. 52(7): p. 2469-2480.
79. S. Mundra, M. C., S. A. Bernal, J. L. Provis., Chloride-induced corrosion of steel rebars in simulated pore solutions of alkali-activated concretes. Cement and Concrete Research, 2017. 100: p. 385-397.
80. Ma, Q., et al., 1,2,3-Triazole derivatives as corrosion inhibitors for mild steel in acidic medium: Experimental and computational chemistry studies. Corrosion Science, 2017. 129: p. 91-101.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A method of making 1-Benzyl-4-Phenyl-1H-1,2,3-triazole (BPT), comprising:
mixing t-BuOH, water, benzyl bromide and sodium azide to form a first mixture;
mixing phenylacetylene, ascorbic acid, sodium hydroxide, and pentahydrated copper sulfate with the first mixture, wherein a precipitate is formed; and
separating the precipitate, wherein the precipitate is BPT.

2. The method of claim 1, wherein phenylacetylene, ascorbic acid, sodium hydroxide, and pentahydrated copper sulfate are mixed to form a second mixture prior to mixing with the first mixture.

3. The method of claim 2, wherein the first mixture and the second mixture are mixed to form a third mixture.

4. The method of claim 3, wherein the first mixture is mixed for at least 1 hour.

5. The method of claim 3, wherein the first mixture is mixed for about 12 to 24 hours.

6. The method of claim 3, wherein the second mixture is mixed for at least 1 hour.

7. The method of claim 3, wherein the second mixture is mixed for about 12 to 24 hours.

8. The method of claim 3, wherein the third mixture is mixed for at least 1 hour.

9. The method of claim 3, wherein the third mixture is mixed for about 12 to 24 hours.

10. The method of claim 1, wherein the yield for producing BPT is greater than 90%.

11. The method of claim 1, wherein the yield for producing BPT is 95% or more.

12. A method of making 1-Benzyl-4-Phenyl-1H-1,2,3-triazole (BPT) comprising the following reaction mechanism:

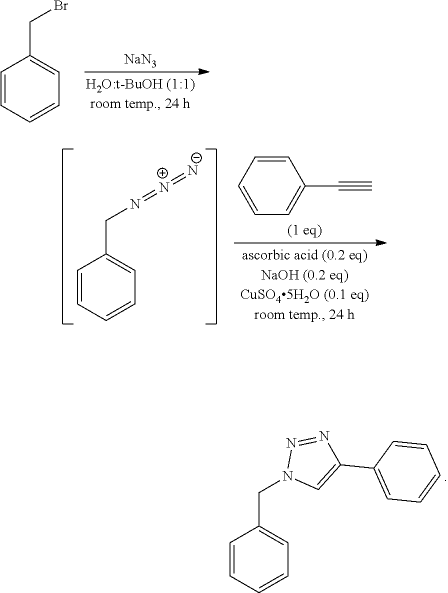

13. A structure comprising: a steel structure having a coating of 1-Benzyl-4-Phenyl-1H-1,2,3-triazole (BPT) disposed on the surface, wherein the BPT was made using the method of claim 1.

14. The structure of claim 13, wherein the coating of BPT has a thickness of about 500 nm to 500 mm.

15. The structure of claim 13, wherein the coating of BPT has a thickness of about 1 mm to about 10 mm.

16. The structure of claim 13, wherein the steel structure is rebar.

17. The structure of claim 16, wherein the rebar disposed within a concrete material.

18. The structure of claim 17, wherein the coating of BPT has the characteristic of inhibiting the corrosion of the steel structure in SPS with 2 M NaCl at BPT concentrations higher than 2 mM.
